(19) United States
(12) Patent Application Publication
(10) Pub. No.: US 2024/0316027 A1
(43) Pub. Date: Sep. 26, 2024

(54) USE OF MARAVIROC IN PREPARATION OF DRUGS FOR TREATMENT OF MUSCLE DEGENERATIVE DISEASES

(71) Applicant: Center for Neuromusculoskeletal Restorative Medicine Limited, Hong Kong (HK)

(72) Inventors: Huating WANG, Hong Kong (HK); Yang LI, Hong Kong (HK)

(21) Appl. No.: 18/592,683

(22) Filed: Mar. 1, 2024

(30) Foreign Application Priority Data
Mar. 3, 2023 (CN) 202310198493.3

(51) Int. Cl. A61K 31/46

(52) U.S. Cl. CPC A61K 31/46

(57) ABSTRACT

Disclosed is the use of Maraviroc in the preparation of drugs for the treatment of muscle degenerative diseases. Demonstrated herein, for the first time, is the outstanding effect of Maraviroc in treating muscle degenerative diseases, such as muscle inflammation, muscle dystrophy and sarcopenia. The underlying mechanism of such use is also disclosed, which provides a knowledge foundation for such novel and effective treatment strategy.

### FIELD OF THE INVENTION

The present invention relates to the field of chemical drugs, and specifically relates to the use of Maraviroc (structure as shown in formula (I)), a geometric isomer thereof, a pharmaceutically acceptable salt, solvate or hydrate thereof, or a pharmaceutical composition thereof in preparation of drugs for treatment of muscle degenerative diseases.

### BACKGROUND OF THE INVENTION

Skeletal muscle refers to muscle in the usual sense, accounting for about 30% to 40% of the human body weight. As one of the most important organs of human beings, skeletal muscle plays a vital role in humans' daily movement, protection, and breathing. However, skeletal muscle is also one of the most vulnerable parts to injuries. For example, muscular dystrophy is a group of genetic disorders characterized by progressive muscle weakness and degeneration. The common and well-known form includes Duchenne muscular dystrophy (DMD), in which muscles undergo degeneration/regeneration, manifested by chronic inflammation and fibrosis. Another age-related muscle degenerative disease is sarcopenia, which is characterized by the progressive loss of muscle mass, strength, and function. It commonly occurs in elderly adults, leading to difficulties with mobility, balance, and overall physical performance decline. In fact, skeletal muscle possesses remarkable self-repair function and can often complete its repair in a short time following acute injuries. Adult muscle stem cells (MuSCs) are indispensable skeletal muscle repair. However, in some chronic muscle degenerative diseases, such as muscle dystrophy and sarcopenia, the function of muscle stem cells is often compromised, leading to impaired muscle repair and muscle function. Muscle stem cell functions are tightly regulated by its niche microenvironment via cellular interactions with various types of cells in the niche such as macrophages and Fibro-adipogenic progenitors (FAPs). Deregulated cellular interactions may cause excessive inflammation and fibrosis in the niche that eventually hinders the functioning of muscle stem cells. Therefore, targeting the stem cell-niche interactions may inhibit the occurrence of inflammation which has a beneficial effect on promoting the function of muscle stem cells.

Maraviroc (MVC) is a small molecule drug developed by Pfizer that can inhibit human immunodeficiency virus type 1 (HIV-1) from entering cells. Its mechanism of action is blocking the interaction between HIV-1 and the chemokine receptor CCR5 on host cells. It was approved by the US FDA and the European Commission in August 2007 and September 2007, respectively, for the treatment of patients with CCR5-tropic HIV-1 infection in combination with other antiretroviral drugs. Specifically, the GP120 protein on the surface of the HIV virus binds to the CCR5 receptor on the host's immune cells, facilitating the entry of the HIV virus into the host's immune cells and thereby destroying the normal function of the immune cells. Maraviroc acts as an antagonist of the CCR5 receptor which can quickly occupy the CCR5 receptor on the surface of immune cells after entering the human body, thereby effectively blocking the binding of GP120, shutting out the HIV virus, and protecting the human immune system from viral infection. Regulatory approvals were granted by the U.S. Food and Drug Administration (FDA) and the European Commission in August and September 2007, respectively, facilitating its utilization in conjunction with other antiretroviral agents for the management of CCR5-tropic HIV-1 infection. In addition to HIV/AIDS, CCR5 has been implicated in other diseases, including cancer, inflammatory and graft-versus-host diseases (GVHDs). Therefore, it was proposed that blocking CCR5 can attenuate the severity or progression of these diseases. For example, CCR5 ligands and other chemokines are increased in synovial fluids during rheumatoid arthritis (RA), resulting in tissue and joint damage. Therefore, blocking CCR5 could reduce inflammation at synovial joints and reduce RA symptoms. However, in a Phase IIa study, Maraviroc showed no efficacy in the treatment of RA.

It is found in the present invention that deleting YY1, a transcription factor in MuSCs from mdx mice (dystrophic mouse model) causes severe dystrophic pathologies such as aggravated inflammation and fibrosis, decreased muscle mass and function, declined life span, to a degree comparable to human DMD patients. Mechanistic investigation revealed that the YY1 deletion causes CCL5 up-regulation in MuSCs and enhances CCL5/CCR5 mediated macrophage recruitment resulting in the increased inflammation and skewed niche composition. It is therefore hypothesized in the present invention that targeting the CCL5-CCR5 axis can attenuate MuSC-macrophage interaction therefore reducing the inflammation in the dystrophic muscle. Furthermore, it is found in the present invention that the CCL3, 4 and 5 are also up-regulated in aging mouse and human muscles along with CCR5, therefore, targeting CCR5 can also be a strategy to ameliorate age-associated sarcopenia.

### SUMMARY OF THE INVENTION

It is an objective of the present invention to provide a Maraviroc (structure as shown in formula (I)), a geometric isomer thereof, a pharmaceutically acceptable salt, solvate or hydrate thereof, or a pharmaceutical composition thereof for use in preparing drugs for the treatment of muscle degenerative diseases.

Accordingly, the objective can be achieved by following the technical solutions of the present invention as follows:

The present invention provides a method for using a compound of formula (I):

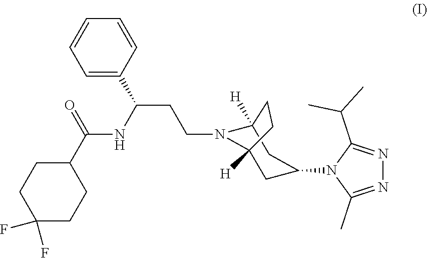 [SMILES: Cc1nnc(C(C)C)n1[C@@H]1C[C@H]2CC[C@@H](C1)N2CC[C@H](NC(=O)C1CCC(F)(F)CC1)c1ccccc1]

or a geometric isomer thereof, or a pharmaceutically acceptable salt, solvate or hydrate thereof, or a pharmaceutical composition comprising one or more ingredients thereof, in preparation of drugs for treatment of muscle degenerative diseases.

In one embodiment, the method is further used in preparation of drugs for treatment of muscle weakening due to aging.

The muscle degenerative diseases specifically refer to skeletal muscle degeneration diseases.

The muscle degenerative diseases include muscle inflammation, muscle dystrophy or sarcopenia.

The compound of formula (I) achieves muscle repair function by blocking CCL5-CCR5 signalling pathway.

The content of the compound of formula (I) or the geometric isomer thereof, or a pharmaceutically acceptable salt, solvate or hydrate thereof is 1%˜99% of the total weight of the pharmaceutical composition.

The pharmaceutical composition further comprises a pharmaceutically acceptable carrier or excipient.

The pharmaceutically acceptable carrier or excipient is selected from the group consisting of solvents, solubilizers, cosolvents, emulsifiers, flavoring agents, olfactory agents, colorants, adhesives, disintegrants, fillers, lubricants, wetting agents, osmotic pressure regulators, pH regulators, stabilizers, surfactants, preservatives or any combinations thereof.

The pharmaceutical composition is a solid preparation, a semi-solid preparation or a liquid preparation.

Preferably, the solid preparation includes a tablet, capsule, granule and/or pill; the semi-solid preparation includes a gel, suppository and/or ointment; and the liquid preparation includes an emulsion, mixture, suspension and/or solution.

Further preferably, the pharmaceutical composition is an injection.

The beneficial effect of the present invention is that the present invention discovers and demonstrates for the first time the outstanding effect of Maraviroc in treating muscle degenerative diseases, such as muscle dystrophy and sarcopenia. The present invention also explores the underlying mechanism of such use, which provides knowledge foundation for such treatment strategy.

### BRIEF DESCRIPTION OF THE DRAWINGS

The features of the invention will be more readily understood and appreciated from the following detailed description when read in conjunction with the accompanying drawings of the preferred embodiment of the present invention, in which:

FIG. 1 illustrates a therapeutic strategy using Maraviroc against Duchenne muscular dystrophy in mice;

FIG. 1A illustrates a comparison of weights in dKO mice compared to Ctrl mice;

FIG. 1B illustrates a comparison of lifespan, loss in weight of tibialis anterior (TA) muscle and thickness of diaphragm (DP) muscle in dKO mice compared to Ctrl mice;

FIG. 1C illustrates a comparison of muscle fiber size in dKO mice compared to Ctrl mice using frozen muscle sections stained with H&E;

FIG. 1D illustrates a comparison of spread of stained fibrotic area (blue) over spread of stained muscle tissue (red) in dKO mice and Ctrl mice using frozen muscle sections stained with Masson Trichrome;

FIG. 2 illustrates data of body weight measurement of the treated mice;

FIG. 3 illustrates a comparison of muscle fiber size in the treated mice using frozen muscle sections stained with H&E;

FIG. 4 illustrates a comparison of spread of stained fibrotic area (blue) over spread of stained muscle tissue (red) in the treated mice using frozen muscle sections stained with Masson Trichrome;

FIG. 5 illustrates a comparison of muscle endurance in the treated mice using a forced training model;

FIG. 6 illustrates a comparison of muscle functions in the treated mice using a voluntary training model;

FIG. 7 illustrates a therapeutic strategy using Maraviroc against sarcopenia in aging mice;

FIG. 8 illustrates a comparison of muscle in the treated aging mice using frozen muscle sections stained with H&E;

FIG. 9 illustrates the comparison of muscle strength and endurance in the treated aging mice by the grip strength measuring and the forced training model;

FIG. 10 illustrates a comparison of main cell proportions in the treated aging mice.

### DETAILED DESCRIPTION OF THE INVENTION

For the purposes of promoting and understanding the principles of the invention, reference will now be made to the embodiments illustrated in the drawings and described in the following written specification. It is understood that the present invention includes any alterations and modifications to the illustrated embodiments and includes further applications of the principles of the invention as would normally occur to one skilled in the art to which the invention pertains.

### EMBODIMENTS

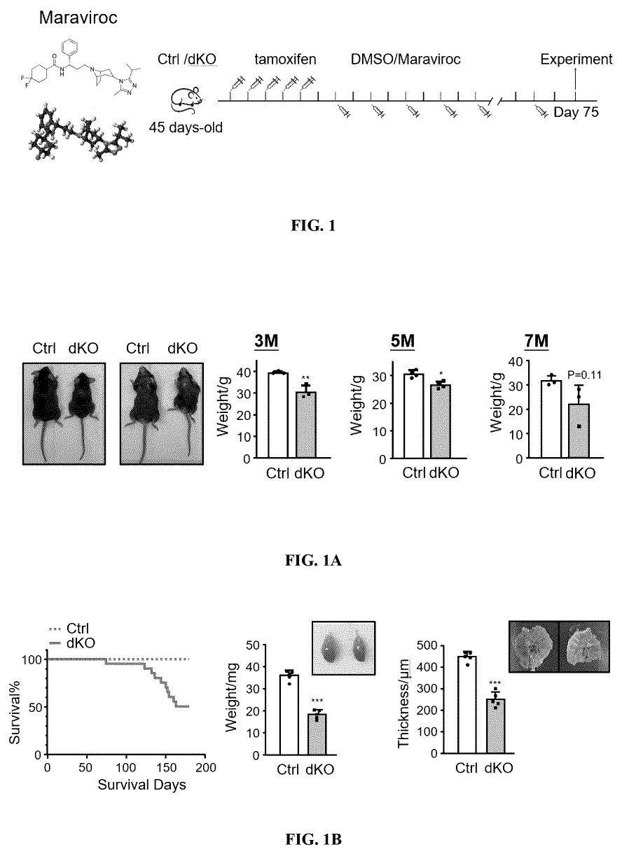

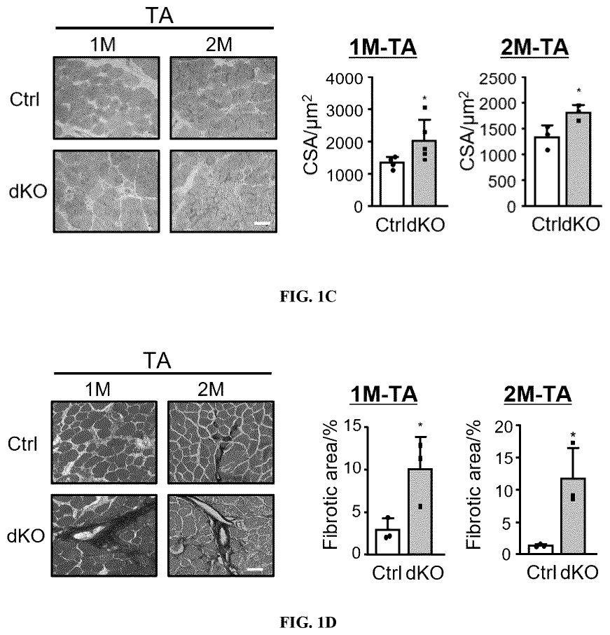

The research object of this invention is based on a mouse model of human Duchenne muscular dystrophy (DMD), a congenital mutation in the muscular dystrophy protein leading to spontaneous periodic muscle damage and repair. Chronic inflammation and fibrosis are typical hallmarks of DMD and may contribute to impaired skeletal muscle regeneration. Mdx mice are commonly used DMD mouse model which, however, shows much milder symptoms compared to human DMD. It is found in the present invention that deleting YY1, a transcription factor in MuSCs from mdx mice (dystrophic mouse model) causes severe dystrophic pathologies such as aggravated inflammation and fibrosis, decreased muscle mass and function, declined life span, to a degree comparable to human DMD patients. These YY1; mdx mice (so called double knock out dKO) display decreased body and muscle weight, severe inflammation and fibrosis, and reduced lifespan. As illustrated in FIGS. 1A-1D, the body weights are continuously monitored and measured at 3M, 5M and 7M intervals and the dKO mice show continuous weight loss compared to Ctrl mice (see FIG. 1A) as time progresses. Furthermore, the reduction in muscle weight (tibialis anterior) and thickness (diaphragm) is also observed over time in contrast to Ctrl mice (see FIG. 1B). The lifespan (or survival rate) of the dKO mice is significantly decreased compared to Ctrl mice (see FIG. 1B). When the muscle morphology is examined, as shown in FIG. 1C, H&E staining is performed on the muscle sections, where the muscle fibers are stained pink and the cell nuclei are stained purple. the cross-sectional area (CSA) of TA muscle fiber in dKO mice is significantly higher than the Ctrl mice, indicating an abnormal hypertrophy in the dKO muscle tissue. Meanwhile, excessive inflammation is observed on the dKO muscles as shown by the amount of immune cells infiltration in the interstitial area (see FIG. 1C), along with aggravated fibrosis (detected by Masson's Trichrome staining, see FIG. 1D). Altogether, the findings demonstrate that the dKO mice are good models for testing the treatment effect of Maraviroc (MVC).

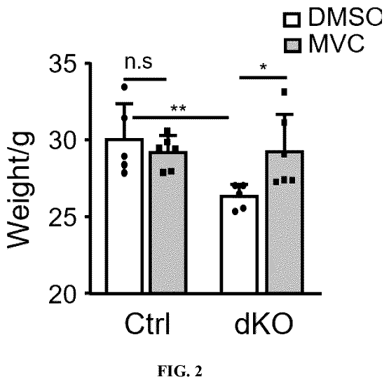

To test the therapeutic effect of Maraviroc on the dystrophic muscles, the above-described Ctrl and dKO mice are treated with dimethyl sulfoxide (DMSO) or Maraviroc respectively. 100 uL 5% DMSO and 100 uL Maraviroc (60 ug dissolved in 5% DMSO) are injected intraperitoneally every two days for one month, as illustrated in FIG. 1, the overall morphology and health state of the dKO mice treated with Maraviroc have improved showing a significantly restored body weight and muscle weight, as illustrated in FIG. 2. This indicates that Maraviroc effectively inhibits muscle loss in dKO mice. In addition, it is also found that injection of Maraviroc has no obvious effect on the body weight of Ctrl mice which is also illustrated in FIG. 2.

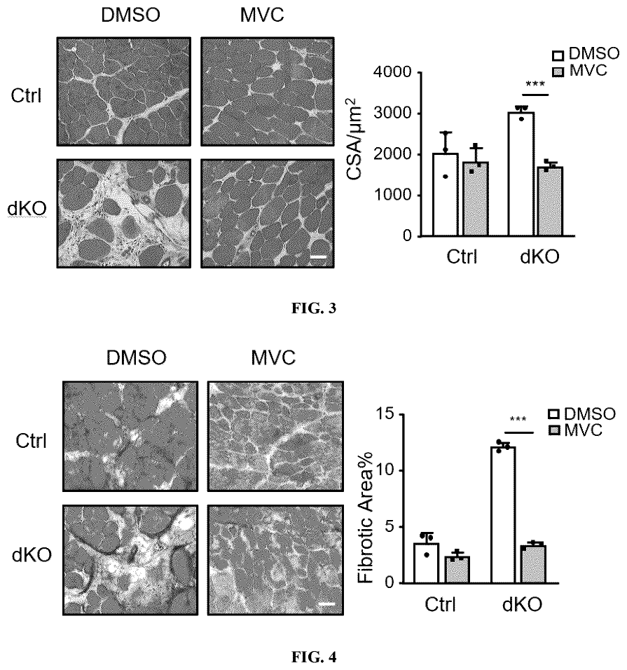

Next, the muscle morphology is evaluated on the above-treated four groups of mice. H&E staining of the TA muscles uncovers that the muscle fibers of dKO mice treated with DMSO show severe impairment in muscle regeneration as evidenced by the abnormal hypertrophy of muscle fiber; a large number of infiltrating immune cells is also observed, indicating that a strong inflammation in the muscle tissue. The Ctrl and dKO muscles treated with Maraviroc show improved morphology with reduced immune cell infiltration and inflammation, as illustrated in FIG. 3. In addition, Masson's Trichrome staining shows that Maraviroc can also significantly alleviate the muscle fibrosis in dKO mice, as illustrated in FIG. 4. Therefore, the above results indicate that Maraviroc can significantly reduce inflammation and fibrosis and improve muscle repair in dystrophic muscle.

Next it is examined if the treatment improves muscle functions by testing the muscle performance in exercises. Both endurance and voluntary exercises are employed. For endurance exercise, mice are first placed in a treadmill machine, where the bottom of the running track is equipped with an electric shock device to force the mice to continue running as illustrated in FIG. 5. The initial speed is set to 10 cm/s and increased by 5 cm/s every two minutes. By recording the running distance until the mice are exhausted, their endurance strength is determined. The results uncover that the dKO mice treated with DMSO show a significant decrease in endurance compared to the Ctrl mice treated with DMSO, but the endurance strength of the dKO mice treated with Maraviroc is significantly higher than that of the dKO mice treated with DMSO, indicating that Maraviroc treatment improves the muscle strength and performance in endurance exercise as illustrated in FIG. 5. For voluntary exercise, the mice are placed in cages equipped with exercise wheels, which are connected to counters that accurately record the distance travelled by the mice on the wheels, as illustrated in FIG. 6. The mice are continuously housed in the cages for one week, and their exercise distance on the wheels is recorded daily. The data records over the course of one week show that the Ctrl mice treated with DMSO or Maraviroc are engaged in a high level of voluntary exercise. dKO mice treated with DMSO show much lower interest in exercise but Maraviroc treatment leads to a consistent increase in running distance over time as illustrated in FIG. 6. Altogether the above results demonstrate that Maraviroc treatment improves muscle functions of the dystrophic mice.

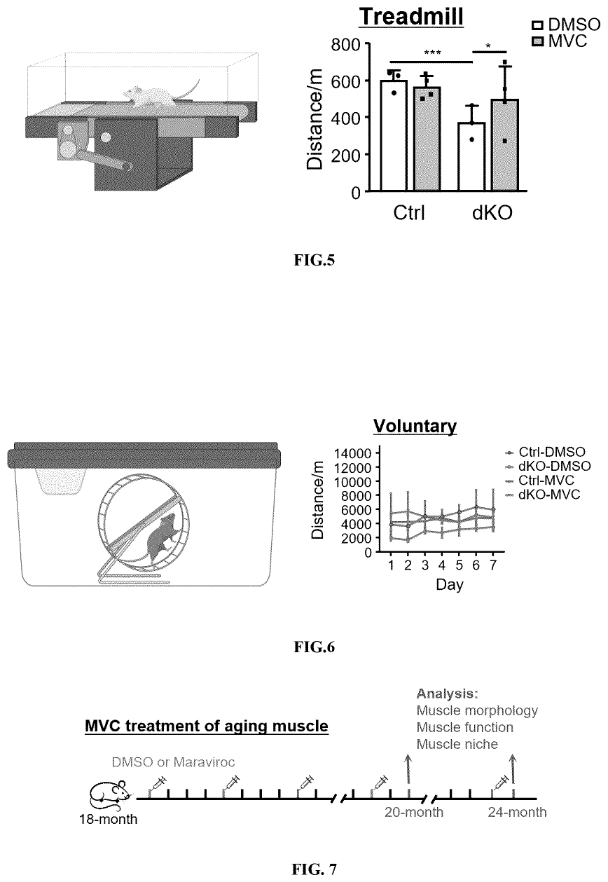

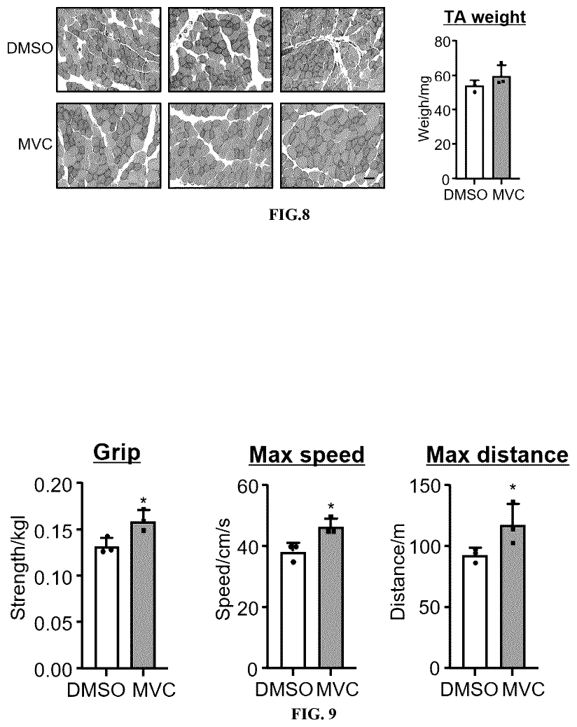

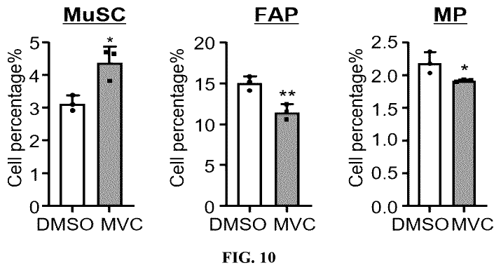

Next, to further illustrate the possible application for treating other muscle degenerative diseases, the effectiveness of Maraviroc is tested in aged mice with sarcopenia. 3 pairs of 16-month-old mice are intraperitoneally injected with vehicle (5% DMSO) or Maraviroc (2.5 mg/kg) twice a week for 6 months, as shown in FIG. 7. H&E staining shows the treatment leads to remarkable improvement of the muscle morphology which includes increased fiber size, attenuated inflammation and restored TA muscle weight as illustrated in FIG. 8. Moreover, muscle functions (which are tested by grip force and treadmill running) are also restored, as illustrated in FIG. 9. Improved muscle stem cell niche is also detected with reduced number of FAPs and macrophages and increased number of MuSCs in FIG. 10. Overall, the Maraviroc treatment delays muscle aging and rejuvenates the mice.

The present invention shows that Maraviroc can effectively alleviate muscle inflammation, fibrosis, and improve MuSC function in both dystrophic and aged mice. It also shows that Maraviroc also significantly improves muscle strength, performance, and physical fitness of the mice, therefore, sufficiently indicating that Maraviroc has an outstanding effect in treating muscle degenerative diseases, such as muscle inflammation, muscle dystrophy and sarcopenia.

The present invention explained above is not limited to the aforementioned embodiment and drawings, and it will be obvious to those having an ordinary skill in the art of the prevent invention that various replacements, deformations, and changes may be made without departing from the scope of the invention.

What is claimed:

1. A method for using a compound of formula (I): 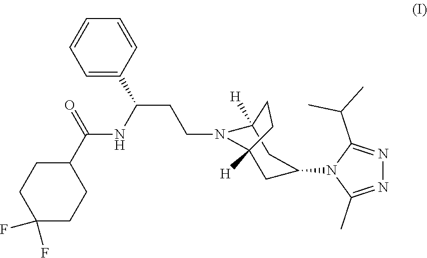 [SMILES: Cc1nnc(C(C)C)n1[C@@H]1C[C@H]2CC[C@@H](C1)N2CC[C@H](NC(=O)C1CCC(F)(F)CC1)c1ccccc1]
or a geometric isomer thereof, or a pharmaceutically acceptable salt, solvate or hydrate thereof, or a pharmaceutical composition comprising one or more ingredients thereof, in preparation of drugs for treatment of muscle degenerative diseases.

2. The method according to claim 1, wherein the method is further used in preparation of drugs for treatment of muscle weakening due to aging.

3. The method according to claim 1, wherein the muscle degenerative diseases specifically refer to skeletal muscle degeneration diseases.

4. The method according to claim 1, wherein the muscle degenerative diseases include muscle inflammation, muscle atrophy or sarcopenia.

5. The method according to claim 1, wherein the compound of formula (I) achieves muscle repair function by blocking CCL5-CCR5 signalling pathway.

6. The method according to claim 1, wherein the content of the compound of formula (I) or the geometric isomer thereof, or a pharmaceutically acceptable salt, solvate or hydrate thereof is 1%˜99% of the total weight of the pharmaceutical composition.

7. The method according to claim 1, wherein the pharmaceutical composition further comprises a pharmaceutically acceptable carrier or excipient.

8. The method according to claim 6, wherein the pharmaceutically acceptable carrier or excipient is selected from the group consisting of solvents, solubilizers, cosolvents, emulsifiers, flavorings agents, olfactory agents, colorants, adhesives, disintegrants, fillers, lubricants, wetting agents, osmotic pressure regulators, pH regulators, stabilizers, surfactants, preservatives or any combinations thereof.

9. The method according to claim 6, wherein the pharmaceutical composition is a solid preparation, a semi-solid preparation or a liquid preparation.

10. The method according to claim 8, wherein the solid preparation includes a tablet, capsule, granule and/or pill; the semi-solid preparation includes a gel, suppository and/or ointment; and the liquid preparation includes an emulsion, mixture, suspension and/or solution.
